(19) United States
(12) Patent Application Publication
(10) Pub. No.: US 2008/0319329 A1
(43) Pub. Date: Dec. 25, 2008

(54) Manual-driven inflation-powered electronic blood pressure measuring apparatus

(75) Inventor: Daniel C.M. Tseng, Taipei County (TW)

Correspondence Address: Joe McKinney Muncy, PO Box 1364, Fairfax, VA, 22038-1364, US

(73) Assignee: K-JUMP HEALTH CO., LTD.

(21) Appl. No.: 11/819,123

(22) Filed: Jun. 25, 2007

(51) Int. Cl. A61B 5/02; A61M 5/178

(52) U.S. Cl. 600/494; 600/490; 600/496; 604/37

(57) ABSTRACT

A manual-driven inflation-powered electronic blood pressure measuring apparatus includes an inflation unit to generate pressurized air by a user through hand squeezing, a power generation unit to receive the pressurized air to generate and output electric power, an electronic blood pressure measurement unit to receive the electric power output from the power generation unit and the pressurized air from the inflation unit to inflate an air pouch, and a sensor to detect a person's blood pressure signals in cooperation with the inflation pouch, and outputs a measurement result through an output unit.

### FIELD OF THE INVENTION

The present invention relates to an electronic blood pressure measuring apparatus and particularly to a manual-driven inflation-powered electronic blood pressure measuring apparatus.

### BACKGROUND OF THE INVENTION

The conventional mercury blood pressure meter has a glass tube containing mercury to measure blood pressure. As the glass tube tends to crack and be damaged under external impact, electronic blood pressure measuring apparatus have been developed. For instance R.O.C. patent No. I275381, entitled “Staged blood vessel pulse measuring apparatus and method based on blood pressure measurement” includes an inflation pouch to surround the arm of a person that is connected to a pressure sensor, an inflation pump connecting to the inflation pouch to inflate the inflation pouch and the pressure sensor can detect a pressure variation signal of the inflation pouch during inflation, a signal processing module which is electrically connected to the pressure sensor to receive the pressure variation signal and analyze the signal to get a pressurizing signal and a resonance signal, a microprocessor which is electrically connected to the signal processing module to receive the pressurizing signal and resonance signal to process a blood pressure parameter to get at least one blood pressure parameter of the person, an air release valve connecting to the inflation pouch to release the pressurized air in a staged manner according to a preset pressure value so that the pressure sensor can detect a staged pressure release signal of the inflation pouch, and the signal processing module analyzes the signal and transmits the signal to the microprocessor to process a pulse parameter to get at least one pulse signal and one pulse parameter, and a display device connecting to the microprocessor to display the blood pressure parameter and the pulse parameter.

The electronic blood pressure measuring apparatus mentioned above has to receive external electric power to function. It cannot function properly at remote locations where no electric power is available or the power is insufficient or unstable. Although users can get electric power by connecting to an external generator or a power storage device such as batteries, an extra generator or power storage is needed. It is not convenient. At the remote locations where few people resided and transportation is difficult, malfunction of the generator or depletion of the power storage could make the electronic blood pressure measuring apparatus useless. Hence how to overcome the aforesaid problems is a big issue in the industry.

### SUMMARY OF THE INVENTION

The primary object of the present invention is to provide an electronic blood pressure measuring apparatus that uses electric power source that is manual and inflation driven so that it can be used to measure blood pressure in environments where power is not available or abnormal.

To achieve the foregoing object the electronic blood pressure measuring apparatus of the invention includes an inflation unit to generate pressurized air by a user through hand squeezing, a power generation unit to receive the pressurized air to generate and output electric power, an electronic blood pressure measurement unit to receive the electric power output from the power generation unit and the pressurized air from the inflation unit to inflate an air pouch, and a sensor to detect a person's blood pressure signal in cooperation with the inflation pouch to output a measurement result through an output unit.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a block diagram of an embodiment of the invention.

FIG. 2 is a block diagram of another embodiment of the invention.

### DETAILED DESCRIPTION OF THE PREFERRED EMBODIMENTS

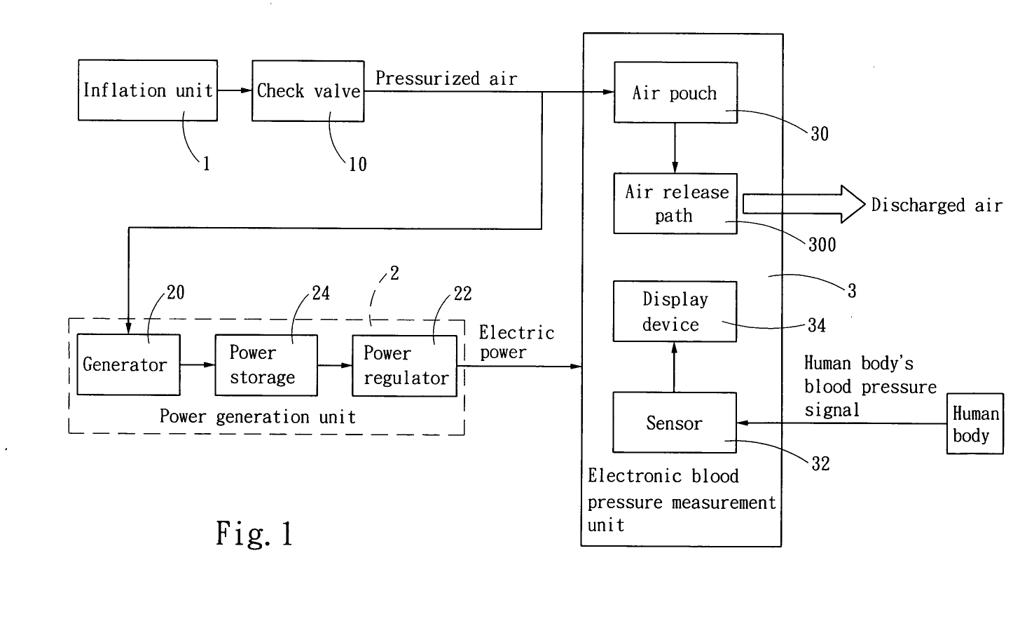

Please refer to FIG. 1 for a block diagram of an embodiment of the invention. The manual-driven inflation-powered electronic blood pressure measuring apparatus of the invention includes:

an inflation unit 1 which generates pressurized air by a user through hand squeezing. The inflation unit 1 has a check valve 10 to prevent the output pressurized air from flowing back to the inflation unit 1;

a power generation unit 2 to receive the pressurized air from the inflation unit 1 to generate and output electric power. In this embodiment the power generation unit 2 includes a generator 20, a power regulator 22 and a power storage 24. The generator 20 is driven by the pressurized air to generate the electric power (such as AC signals). The power regulator 22 may be a rectifier, a voltage stabilizer or a combination thereof to regulate the electric power generated by the generator 20. The power storage 24 stores the electric power generated by the power generation unit 2, and may be a capacitor which also can regulate the electric power; and

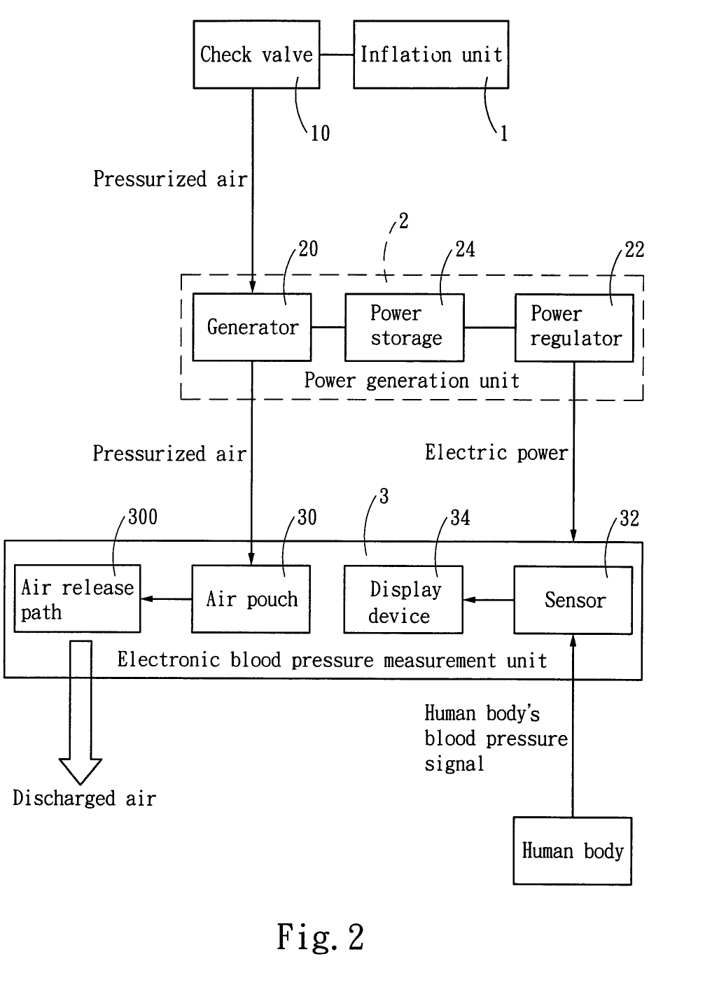

an electronic blood pressure measurement unit 3 to receive the electric power output from the power generation unit 2 and inflate an air pouch 30 with the pressurized air generated by the inflation unit 1, and through the air pouch 30 and a sensor 32 to detect a person's blood pressure signal (commonly called pulse), and output a measurement result through an output unit. In this embodiment the output unit is a display device 34. The air pouch 30 has an air release path 300 to discharge the air in the air pouch 30 after the measurement is finished. In FIG. 1, the electronic blood pressure measurement unit 3 and the power generation unit 2 receive the pressurized air from the inflation unit 1, and the pressurized air is in a parallel coupling fashion. FIG. 2 shows another embodiment in which the electronic blood pressure measurement unit 3 receives the pressurized air of the inflation unit 1 through the power generation unit 2, and the pressurized air in the electronic blood pressure measurement unit 3 and the power generation unit 2 is in a serial coupling fashion.

It is to be noted that in the embodiments set forth above, the power generation unit 2 and the electronic blood pressure measurement unit 3 are conventional devices commonly known. They have many different circuit designs, and are not the focus of the invention and also not the limitation of the invention. Moreover, the output unit such as the display device 34, in addition to delivering measurement result by displaying, may also be in other types to indicate the measurement result through other multimedia means such as turning on and off of light, blinking of light, voice announcement, audio broadcasting, or the like.

In short, the invention drives the power generation unit 2 through the pressurized air of the inflation unit 1 so that the electronic blood pressure measurement unit 3 can measure blood pressure in environments where external electric power is not available or abnormal. Thus it overcomes the problem of the conventional electronic blood pressure measurement apparatus that cannot function properly at remote locations where electric power is not available, insufficient or abnormal. It also resolves the inconvenience of carrying an extra portable power generator or power storage that happens to the conventional electronic blood pressure measurement apparatus. Thus it offers a significant improvement over the conventional techniques.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A manual-driven inflation-powered electronic blood pressure measuring apparatus, comprising:
an inflation unit to generate pressurized air by a user through hand squeezing;
a power generation unit to receive the pressurized air from the inflation unit and generate and output electric power; and
an electronic blood pressure measurement unit to receive the electric power output from the power generation unit and inflate an air pouch with the pressurized air generated by the inflation unit, and detect a person's blood pressure signal through the air pouch and a sensor, and output a measurement result through an output unit.

2. The manual-driven inflation-powered electronic blood pressure measuring apparatus of claim 1, wherein the power generation unit has a generator to receive the pressurized air to generate AC power signals.

3. The manual-driven inflation-powered electronic blood pressure measuring apparatus of claim 1, wherein the power generation unit has a power regulator to regulate the electric power generated by the power generation unit.

4. The manual-driven inflation-powered electronic blood pressure measuring apparatus of claim 3, wherein the power regulator is a rectifier.

5. The manual-driven inflation-powered electronic blood pressure measuring apparatus of claim 3, wherein the power regulator is voltage stabilizer.

6. The manual-driven inflation-powered electronic blood pressure measuring apparatus of claim 1, wherein the power generation unit has a power storage to store the electric power generated by the power generation unit.

7. The manual-driven inflation-powered electronic blood pressure measuring apparatus of claim 1, wherein the air pouch has an air release path.

8. The manual-driven inflation-powered electronic blood pressure measuring apparatus of claim 1, wherein the inflation unit has a check valve.

9. The manual-driven inflation-powered electronic blood pressure measuring apparatus of claim 1, wherein the output unit is a display device.

10. The manual-driven inflation-powered electronic blood pressure measuring apparatus of claim 1, wherein the output unit is a lighting device which turns on and off light to output the measurement result.

11. The manual-driven inflation-powered electronic blood pressure measuring apparatus of claim 1, wherein the output unit is a lighting device which generates blinking light to output the measurement result.

12. The manual-driven inflation-powered electronic blood pressure measuring apparatus of claim 1, wherein the output unit is a sound output device which broadcasts voice to output the measurement result.

13. The manual-driven inflation-powered electronic blood pressure measuring apparatus of claim 1, wherein the output unit is a sound output device which makes audio broadcast to output the measurement result.

14. The manual-driven inflation-powered electronic blood pressure measuring apparatus of claim 1, wherein the pressurized air received by the electronic blood pressure measurement unit and the power generation unit are coupled in a parallel fashion.

15. The manual-driven inflation-powered electronic blood pressure measuring apparatus of claim 1, wherein the electronic blood pressure measurement unit receives the pressurized air from the inflation unit through the power generation unit, and the pressurized air received by the electronic blood pressure measurement unit and the power generation unit are coupled in a serial fashion.
